(19) United States
(12) Patent Application Publication
(10) Pub. No.: US 2024/0001135 A1
(43) Pub. Date: Jan. 4, 2024

(54) METHODS AND SYSTEMS FOR IDENTIFICATION OF TREATMENT TARGETS

(71) Applicant: Magnus Medical, Inc., Burlingame, CA (US)

(72) Inventors: Shan H. SIDDIQI, Hoffman Estates, IL (US); Brandon S. BENTZLEY, Half Moon Bay, CA (US); Armani PORTER, Pikesville, MD (US)

(21) Appl. No.: 18/117,350

(22) Filed: Mar. 3, 2023

Related U.S. Application Data
(60) Provisional application No. 63/369,729, filed on Jul. 28, 2022.
(60) Provisional application No. 63/316,345, filed on Mar. 3, 2022.

(51) Int. Cl. A61N 2/00

(52) U.S. Cl. CPC A61N 2/006

(57) ABSTRACT

Described herein are methods and systems for the identification and selection of a neurostimulation target for treating neurological or psychiatric disorders in a subject using functional connectivity networks. The methods and systems may analyze the connectivity or synchrony between deeper brain regions of interest that have been divided into parcels with surface regions of the brain. The methods and systems generally reduce the computational complexity of the targeting algorithm, which in turn may result in a more reliable determination of the neurostimulation targets. Additionally, the methods and systems may personalize the target for neurostimulation, which may improve the overall efficacy of the neurostimulation treatment. Methods and systems that use a decision algorithm to calculate a weight value for a plurality of seed-circuit pairs, and determine a neurostimulation target based on the seed-circuit pair having the greatest weight value are also described herein.

### CROSS-REFERENCE TO RELATED APPLICATIONS

This application claims priority to U.S. Provisional Application No. 63/316,345, filed Mar. 3, 2022, and U.S. Provisional Application No. 63/369,729, filed Jul. 28, 2022, each of which is hereby incorporated by reference in its entirety.

### TECHNICAL FIELD

This application relates generally to the field of neurostimulation for treating a neurological or psychiatric disorder. More specifically, the application relates to methods for identification of a neurostimulation target using functional connectivity networks. Systems for identifying the neurostimulation targets are also described herein.

### BACKGROUND

Transcranial Magnetic Stimulation (TMS) is a non-invasive medical procedure where strong magnetic fields are utilized to stimulate specific areas of an individual's brain in order to treat medical conditions such as depression and neuropathic pain. Since TMS coils are incapable of focally stimulating the deep brain structures often associated with neurological or psychiatric disorders, functional connectivity studies that link surface regions of the brain to deeper regions are often used to determine neurostimulation targets. Stimulation of these surface regions thus potentially bypasses the need for deep brain stimulation.

Generating personalized neurostimulation targets may improve treatment outcomes for individuals. However, existing methods and systems for target personalization, including methods and systems for functional connectivity-based target identification, suffer from several limitations, including reliance upon single brain seed regions (that is, reliance upon a single region in the brain with respect to which functional connectivity is determined), the assumption that these seed regions can be localized reliably across multiple patients, the assumption that the seed regions serve the same functions across multiple patients, the lack of a built-in metric of internal reliability, and low reproducibility.

Accordingly, it would be useful to have other methods and systems for determining personalized targets for neurostimulation when treating neurological or psychiatric disorders.

### SUMMARY

Described herein are methods and systems for the identification of a neurostimulation target for treating neurological or psychiatric disorders in a subject using functional connectivity networks. The methods and systems may analyze the connectivity or synchrony between brain regions of interest that have been divided into parcels and other brain regions within a search space that is known to be accessible with a given neurostimulation modality such as TMS. In some variations, the brain regions of interest may be brain regions that are potential neurostimulation targets for treatment but are less accessible with a given modality such as TMS. In some variations, the brain regions of interest may be brain regions that are potential neurostimulation targets for treatment but are too distributed, too large, or too small to effectively stimulate with a given modality such as TMS.

The methods and systems described herein generally reduce the computational complexity of the targeting algorithm, which in turn may result in a more reliable and/or less computationally-intensive determination of the neurostimulation targets. Additionally, the methods and systems may personalize the target for neurostimulation, which may improve the overall efficacy of the neurostimulation treatment. The psychiatric disorders that may be treated with the targeted stimulation include without limitation, depression, anxiety, post-traumatic stress disorder (PTSD), obsessive-compulsive disorder (OCD), addictions, substance use disorders, bipolar disorder, and schizophrenia. Exemplary neurological disorders that may be treated with the targeted neurostimulation include without limitation, Parkinson's disease, essential tremor, stroke, epilepsy, traumatic brain injury, migraine headache, cluster headache, chronic pain, and consequences of stroke.

The methods for identification of a neurostimulation target described herein generally include obtaining functional neuroimaging data of a brain of the subject, where the functional neuroimaging data describes neuronal activation (by way of example as indicated by oxygenation within the brain), selecting a region of interest within the subject's brain, dividing the region of interest into a plurality of parcels, determining a peak connectivity site for each of the plurality of parcels based on the functional neuroimaging data, and determining a neurostimulation target based on the plurality of peak connectivity sites.

Peak connectivity sites may be characterized by a pattern of neuronal activity having a high degree of synchrony or anti-synchrony to a pattern of neuronal activity in the corresponding parcel. In some embodiments, the peak connectivity site may be located outside of the region of interest. In some embodiments, the region of interest may be a first cortical region and the neurostimulation target may be a second cortical region that is anatomically smaller relative to the first cortical region. In certain embodiments, the region of interest may be a first cortical region and the neurostimulation target may be a second cortical region that is anatomically less distributed relative to the first cortical region. In certain embodiments, the region of interest may be connected to a targeting seed or seed region. A reference circuit may connect a region of interest to a seed region.

The methods for identification of a neurostimulation target described herein may further include using an algorithm based on at least one weighting factor. The method may comprise obtaining functional neuroimaging data of a region of interest (ROI) in a brain of the subject, selecting at least one seed region within the brain of the subject, and pairing the at least one seed region with a plurality of brain circuits to generate a plurality of seed-circuit pairs. A computer-implemented algorithm may be applied to the plurality of seed-circuit pairs that calculates a weight value for each of the plurality of seed-circuit pairs based on a plurality of criteria. The neurostimulation target may then be determined based on the seed-circuit pair having the greatest weight value.

The functional neuroimaging data may comprise data from at least one of functional magnetic resonance imaging (fMRI), functional near infrared spectroscopy (fNIRS), doppler ultrasound, focused ultrasound, diffusion tensor imaging, diffuse optical tomography, electroencephalography, and magnetoencephalography.

The at least one ROI may be the subgenual cingulate cortex, anterior insula, nucleus accumbens, medial prefrontal cortex, or a combination thereof. In one variation, the at least one ROI may comprise the subgenual cingulate cortex. The number of regions of interest selected may be at least one, two, three, four, five, or more regions.

Each of the plurality of brain circuits may be associated with a brain network. In some variations, the brain network may be a canonical network or a data-driven network. In some variations, the canonical network may comprise a dorsal attention network, a ventral attention network, a frontoparietal network, a default mode network, or a cognitive control network.

The plurality of brain circuits may comprise at least one, two, three, four, five, or more brain circuits. In some variations, the plurality of brain circuits may comprise at least a depression circuit. In one variation, the seed-circuit pair may comprise a dorsolateral prefrontal cortex (DLPFC) and a depression circuit. The plurality of seed-circuit pairs may range from two seed-circuit pairs to 25 seed-circuit pairs, including all values and sub-ranges therein. For example, the number of seed-circuit pairs may be 2, 3, 4, 5, 6, 7, 8, 9, 10, 11, 12, 13, 14, 15, 16, 17, 18, 19, 20, 21, 22, 23, 24, or 25. In some instances, it may be useful to analyze 25 seed-circuit pairs.

The plurality of criteria may include a measurement of connectivity between the at least one seed region and each of the plurality of brain circuits of the plurality of seed-circuit pairs, a confidence value, one or more clinical features, a reliability value, or combinations thereof. The one or more clinical features may comprise a duration of depression, severity of depression, family history of a depressive disorder, history of substance abuse, post-traumatic stress disorder, general anxiety disorder, schizophrenia, obsessive-compulsive disorder, bipolar disorder, or combinations thereof.

The methods and systems may further comprise delivering neurostimulation to the neurostimulation target. The neurostimulation may be used to treat a psychiatric disorder selected from the group consisting of depression, anxiety, post-traumatic stress disorder (PTSD), obsessive-compulsive disorder (OCD), addiction, substance use disorders, bipolar disorder, schizophrenia, and a combination thereof. The neurostimulation may be used to treat a neurological disorder selected from a group consisting of Parkinson's disease, essential tremor, stroke, epilepsy, traumatic brain injury, migraine headache, cluster headache, chronic pain, consequences of stroke, and combinations thereof.

Systems for identifying and treating a neurostimulation target are also described herein. The systems generally include a computer programmed to implement one or more embodiments of the above-noted methods. The system may also comprise a communications interface configured to receive data comprising functional neuroimaging data of the brain of the subject, where the functional neuroimaging data describes neuronal activation within the brain, a memory storing a set of instructions, and one or more processors that are configured to, responsive to the set of instructions: select a region of interest within the subject's brain; divide the region of interest into a plurality of sub-parcels; determine a peak connectivity site for each of the plurality of sub-parcels based on the functional neuroimaging data, wherein the peak connectivity site is characterized by a pattern of neuronal activity having a high degree of synchrony or anti-synchrony to a pattern of neuronal activity in the corresponding sub-parcel; and determine a neurostimulation target based on the plurality of peak connectivity sites.

In some variations, the one or more processors are configured to, responsive to the set of instructions, select at least one seed region within the brain of the subject, pair the at least one seed region with a plurality of brain circuits to generate a plurality of seed-circuit pairs, and apply an algorithm to the plurality of seed-circuit pairs. The algorithm may calculate a weight value for each of the plurality of seed-circuit pairs based on a plurality of criteria, and determine a neurostimulation target based on the seed-circuit pair having the greatest weight value.

The system may be further configured to deliver neurostimulation in various ways. For example, in some instances the system may include a transcranial magnetic stimulation coil configured to deliver neurostimulation to the neurostimulation target. In other instances, the system may include a transducer configured to deliver ultrasound energy to the neurostimulation target. The ultrasound energy may be focused ultrasound energy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart describing an exemplary FCN Targeting method.

FIG. 2 is a flowchart that depicts an exemplary FCN Targeting method for determining a neurostimulation target for treating depression in a subject.

FIG. 3 shows the portion of a subject's brain presumed to be the Dorsal Attention Network.

FIG. 4 shows a portion of a subject's brain presumed to be the Dorsal Attention Network as shown in FIG. 3, following parcellation into parcels.

FIGS. 5A-5D depict an exemplary Dorsal Attention Network parcel (left) and coordinates of the peak connectivity site (right) corresponding to the given Dorsal Attention Network parcel.

FIG. 6 shows a plurality of peak connectivity sites, each corresponding to one of a plurality of Dorsal Attention Network parcels.

FIG. 7 shows a neurostimulation target based on an average of the peak connectivity sites shown in FIG. 6.

FIG. 8 schematically shows a computer system programmed or otherwise configured to execute various aspects of an FCN Targeting method in accordance with an embodiment of the disclosure.

FIG. 9 schematically shows a computer system programmed or otherwise configured to execute various aspects of an FCN Targeting method in accordance with another embodiment of the disclosure.

FIG. 10A is a flow chart that depicts an exemplary decision algorithm for identifying a neurostimulation target in a patient using a plurality of seed-circuit pairs and a plurality weighting factors (criteria).

FIG. 10B is a flow chart describing how the algorithm in FIG. 10A applies a first weighting factor (connectivity) to each seed-circuit pair.

FIG. 10C is a flow chart describing how the algorithm in FIG. 10A applies a second weighting factor (confidence) to each seed-circuit pair.

FIG. 10D is a flow chart describing how the algorithm in FIG. 10A applies a third weighting factor (reliability) to each seed-circuit pair.

FIG. 10E is a flow chart describing how the algorithm in FIG. 10A applies a fourth weighting factor (clinical features).

FIG. 11 is a flow chart describing how the weighting factors of FIG. 10A are combined for each seed-circuit pair to produce a ranked list of pairs.

### DETAILED DESCRIPTION

Described herein are methods and systems for the identification of neurostimulation targets for a subject. The method may be referred to as a Functional Connectivity Network Targeting (“FCN Targeting”) method. The methods may be used to treat psychiatric disorders such without limitation depression, anxiety, post-traumatic stress disorder (PTSD), obsessive-compulsive disorder (OCD), additions, substance use disorders, bipolar disorder, and schizophrenia, and neurological disorders such as without limitation Parkinson's disease, essential tremor, stroke, epilepsy, traumatic brain injury, migraine headache, cluster headache, chronic pain, and consequences of stroke

In some instances, a plurality of small neuroanatomical volumes (referred to herein as “parcels”) may be used that are parcels of a larger region of interest (ROI), rather than a single seed to determine a neurostimulation target. The use of a plurality of ROI parcels may obviate reliance on the assumption that a specific, small-volume parcel can be localized in an individual patient, when the location may in fact be variant between individuals. In any given patient, most of ROI parcels may be part of the network of interest (within-network parcel), while some may not (outside-network parcel). However, the connectivity of the outside-network parcels will generally be randomly distributed across other networks and act as background noise such that its contribution to the overall determination of peak connectivity sites may be relatively weak in comparison to the within-network parcels.

Additionally, the methods and systems described herein may be less computationally intensive, thus reducing the computation time as well as improving the efficiency of the computation. For example, one method that has been explored to determine neurostimulation targets in the past employs whole-brain classifiers. This method typically requires computation of connectivity with tens of thousands (or hundreds of thousands) of distinct brain voxels, followed by dimensionality reduction. In contrast, embodiments of the methods described herein include computation of connectivity with a limited number of parcels (by way of example, around 5 to 100 parcels), which is less computationally intensive compared to the whole-brain classifier method.

Furthermore, machine learning algorithms for analyzing functional neuroimaging data are generally trained on data obtained from healthy subjects, which are more readily available than data from subjects with a given brain pathology of interest. However, there is an ever-present concern that algorithms trained with data from healthy brains are sub-optimal for analyzing data obtained from subjects with brain pathologies. Thus, embodiments of the methods described herein may be configured to determine neurostimulation sites for a subject having a brain pathology based on the subject's own functional neuroimaging data, thus personalizing the neurostimulation target.

### Methods

### Determining Neurostimulation Targets Based on Peak Connectivity

The FCN Targeting method and its variations described herein may have characteristics beneficial to the targeting of neurostimulation sites for treating a neurological or psychiatric disorder. As previously mentioned, the FCN Targeting method may improve the efficiency of connectivity computation and help personalize the brain target for neurostimulation. In general, the FCN Targeting method comprises obtaining functional neuroimaging data of a brain of the subject, where the functional neuroimaging data describes neuronal activation within the brain, selecting a region of interest within the subject's brain, dividing the region of interest into a plurality of parcels, and determining a peak connectivity site for each of the plurality of parcels based on the functional neuroimaging data The peak connectivity site may be characterized by a pattern of neuronal activity having a high degree of correlation or anti-correlation to a pattern of neuronal activity in the corresponding parcel. A neurostimulation target may then be determined based on the plurality of peak connectivity sites.

The functional neuroimaging data may be obtained from a functional neuroimaging device, or a data storage unit. The functional neuroimaging data may be in one of the following modalities, without limitation: magnetic resonance imaging (“MRI”) by way of example functional MRI (“fMRI”), diffuse optical imaging (“DOI”) (e.g. diffuse optical tomography), computer-aided tomography (“CAT”), event-related optical signal (“EROS”) imaging, magnetoencephalography (“MIEG”), positron emission tomography (“PET”) by way of example single-photon emission computerized tomography (“SPECT”), electroencephalography (“EEG”), and/or functional near-infrared spectroscopy (“fNIRS”). The functional neuroimaging data may be of the entire brain, or of pre-defined regions within the brain. In one embodiment, the functional neuroimaging data may be obtained from fMRI data. In another embodiment, the functional neuroimaging data may be combined with, derived from, or partially derived from structural connectivity data such as structural MRI or diffusion tensor imaging (DTI), for example by estimating a Bayesian prior of functional connectivity using structural connectivity data in order to refine estimates of functional connectivity, activity, or other functional neuroimaging data.

In certain embodiments, the functional neuroimaging data may comprise data sufficient for a four-dimensional (“4D”) reconstruction of neuronal activity within the imaged brain region(s) with three-dimensional (“3D”) space over a period of time. The 4D reconstruction may be characterized by a voxel resolution, a scanning frequency, and a duration. The voxel resolution may be, for example and without limitation, 0.5 mm to 10 mm in each dimension. The scanning frequency may be, for example and without limitation, under 1 millisecond (for example, in the case of EEG) or as high as 10 seconds (for example, in the case of certain types of MRI). The scanning duration may, for example and without limitation, range from 2-3 minutes to several hours.

The region of interest in the brain (ROI), may be any suitable region. In certain embodiments, the ROI may be a region in the cerebral cortex such as specific subdivisions of the prefrontal cortex (“PFC”) or a region in the limbic system such as the amygdala. In certain embodiments, the ROI may be a brain network associated with a neurological or psychiatric indication based on previous studies, and may therefore be referred to herein as a “disorder-associated network”. The disorder-associated network may be the dorsal attention network (“DAN”), whose abnormal activity has been previously associated with depression. The disorder-associated network may be the anxiosomatic network (“ASN”), whose abnormal activity has been previously associated with anxiety. Other examples of disorder-associated networks may include the limbic network, the ventral attention network, or the cingulo-opercular network, each of which is implicated in various normal functions of the human brain, and each of which (when network pathophysiology such as reduced connectivity or changes in activation is present) has been implicated in various abnormal functions such as neurological and/or psychiatric disorders.

The DAN, also known anatomically as the dorsal frontoparietal network (“D-FPN”), is a large-scale brain network of the human brain that is primarily composed of the intraparietal sulcus (IPS) and frontal eye fields (FEF) and may also include the middle temporal region (MT+), superior parietal lobule (SPL), supplementary eye field (SEF), ventral premotor cortex, and dorsolateral prefrontal cortex. Pathophysiology such as reduced connectivity or changes in activation of the DAN has been linked with various neurological and/or psychiatric disorders. For example, reduced connectivity within the dorsal and ventral attention networks has been linked to higher levels of attention deficit hyperactivity disorder symptoms; reduced connectivity between the DAN and the frontoparietal control network is associated with major depressive disorder; and overactivation of the DAN has been observed in patients with schizophrenia.

The ASN was recently defined based on the connectivity of TMS sites that relieve anxiety and somatic symptoms. The network includes the dorsomedial prefrontal cortex (DMPFC), ventromedial prefrontal cortex (VMPFC), posterior cingulate cortex (PCC), and medial temporal lobes (MTL). Pathophysiology of this network has been implicated in symptoms of neurological and/or psychiatric disorders, such as in anxious and somatic symptoms of depression.

The limbic network is a network in the brain which may include the amygdala, thalamus, hypothalamus, hippocampus, and paralimbic structures such as entorhinal cortex, temporal cortex, and anterior cingulate cortex. Healthy functioning of this network is important for memory, emotion, behavior, and control of the senses. Pathophysiology or disruption of this network is linked to disorders such as temporal lobe epilepsy, dementia, anxiety, bipolar disorder, schizophrenia, depression, autism, and disorders of aggressive or impulsive behavior.

The ventral attention network is a network in the brain which may include the temporoparietal junction (inferior parietal lobule/superior temporal gyrus) and ventral frontal cortex (inferior frontal gyrus/middle frontal gyrus), and may be right-lateralized. This network is thought to act to interrupt other attentional systems, directing attention to relevant events, and pathophysiology or disruption of this network may be linked to disorders such as attention deficit hyperactivity disorder (ADHD), autism or autistic spectrum disorder (ASD), or spatial neglect (for instance, as a symptom of stroke).

The cingulo-opercular network is a network in the brain which may include the dorsal anterior cingulate cortex, anterior insula/frontal operculum, anterior thalamus, putamen, cerebellum, and anterior prefrontal cortex. This network is thought to control goal-directed behavior, and pathophysiology or disruption of this network may be linked to disorders such as schizophrenia, depression, and symptoms of traumatic brain injury.

In alternative interpretations of the disorder-associated networks described here, the networks described herein may contain additional brain structures other than those listed, and/or may not include any of the listed brain structures. Other disorder-associated networks may include without limitation the frontoparietal network, the visual network, the sensorimotor network, and the default mode network.

Directly stimulating a disorder-associated network may not be practicable for a number of reasons, such as neuroanatomical diffuseness and difficult access due to, by way of example, anatomical depth and thus distance from the brain surface. By way of example, the DAN is widespread and covers disparate portions of the brain, as the IPS, FEP, MT+, SPL, and SEF are not adjacent brain regions. As such, attempting to stimulate most or all of the brain regions comprised in a disorder-associated network, in addition to the practical difficulty of how to effectively stimulate a wide area of neural tissue, may create safety and other complications. Therefore, it may be beneficial to locate a neurostimulation target whose activity is more neuroanatomically compact and more easily targeted, and whose stimulation would be expected to robustly modulate the activity the disorder-associated network.

In accordance with embodiments of the FCN Targeting method, the ROI may be divided (“parcellated”) into a plurality of parcels ranging from about 5 to about 100 parcels, including all values and sub-ranges therein. For example, the regions of interest may be divided into 5, 15, 20, 25, 30, 35, 40, 45, 50, 55, 60, 65, 70, 75, 80, 85, 90, 95, or 100 parcels. Although embodiments of the method with a number of parcels less than or equal to 100 may be useful to perform targeting with efficient use of computing resources, in other embodiments, the number of parcels may be larger than 100. Each parcel may be divided in accordance with a predetermined voxel volume, or in accordance with a published brain parcellation map or a brain atlas. Examples of a published parcellation map or brain atlas include a Schaefer 2018 parcellation map, the Gordon 2016 parcellation map, Mindboggle 101, the Consensual Atlas of Resting-State Networks (CAREN), MICCAI 2012, the Brainnetome Atlas Parcellation, the Harvard Oxford Cortical/Subcortical Atlas, AICHA (Atlas of Intrinsic Connectivity of Homotopic Areas), the Hammersmith Atlas, the Yeo Functional Parcellation, and the JuBrain/Juelich Atlas.

A peak connectivity site (“PCS”) may then be determined for each parcel of the plurality of parcels. For example, in a case where a given ROI is parcellated into 30 parcels, the method may result in the designation of 30 PCSs, one PCS for each of the 30 parcels. Each PCS may be characterized by a set of brain anatomy coordinates of the subject.

The PCS for a given parcel may be determined based on a degree of synchrony of neuronal activity, as determined by the functional neuroimaging, with a given parcel. In some instances, the PCS has a high degree of synchrony with the corresponding parcel. In other instances, the PCS has a high degree of anti-synchrony with the corresponding parcel.

In certain embodiments, the determination of the PCS for a given parcel based on a degree of synchrony of neuronal activity may be performed as follows. Using fMRI, the spontaneous fluctuations in activity of brain regions over time are measured. The spontaneous activity within the parcel is averaged, yielding a time series of activity for the parcel. From the fMRI data, a time series of activity for every voxel in the brain is also known. Pearson correlations are then computed between the time series for the parcel and the time series for every other voxel in the brain. This correlation analysis yields a map of synchrony of the parcel with every other voxel in the brain. Using this map, the PCS can be determined, for instance by determining the voxel having absolute peak Pearson correlation with the parcel, or by determining the cluster of voxels having strongest Pearson correlation(s) with the parcel.

In certain embodiments, the PCS may be determined by determining a degree of synchrony between a subcomponent of a parcel and other subcomponents within the parcel, or by determining a degree of synchrony between a subcomponent of the parcel and subcomponents within other parcels.

The PCS may be selected from within a candidate target region in the brain. The candidate target region may be selected based on the selection of the ROI, and/or the selection of a brain disorder that the subject is diagnosed with or a symptom or cluster of symptoms that are desired to be treated. In certain embodiments, the candidate target region may be overlapping, partially overlapping or non-overlapping with the given parcel. In certain embodiments, the candidate target region may be overlapping, partially overlapping or non-overlapping with the ROI as a whole. In other embodiments, the candidate target region may be a brain region that is known, based on prior functional imaging, electrophysiological or anatomical studies, to be expected to exhibit neural connectivity with the ROI or portions thereof, and is also anatomically accessible to neurostimulation. In other embodiments, the candidate target region may be the entire brain or a set of brain regions. A brain region that is anatomically accessible to neurostimulation may be characterized by being a relatively superficial region of the brain, by way of example, a portion of the cortex that is within a threshold depth from the skull. By way of example, where the ROI is a dorsal attention network, the candidate target region may be the dorsolateral pre-frontal cortex (“DLPFC”). By way of another example, where the ROI is an anxiosomatic network, the candidate target region may be the dorsomedial pre-frontal cortex (“DMPFC”).

In certain embodiments, the reliability of the designation of respective peak correlation site for each seed may be assessed. Optionally, the reliability may be assessed by dividing the functional neuroimaging data into two or more data subsets, repeating the designation of the peak correlation sites with each data subset, and comparing PCSs designated based on each data subset. By way of example, a coordinate C₁ for a peak correlation site may be determined for a parcel SP₁ based on each of data sets DS1 and DS2, thus creating coordinate C_{1,1} based on data set DS1 and coordinate C_{1,2} based on data set DS2. The process may be repeated for each of N parcels SP_{N} to generate N coordinates C_{N,1} based on data set DS1 and another N coordinates C_{N,2} based on data set DS2. Then the distance between each pair of coordinates C_{N,1} and C_{N,2} may be calculated, and the PCSs may be determined to be reliable is the average distance is below a predetermined threshold.

Additionally or alternatively, the reliability of the designation of the peak correlation sites may be assessed through a Monte Carlo simulation in which the designation of the PCSs are repeated using N randomly-selected seeds throughout the brain rather than the N parcels divided from the ROI.

Once the PCSs are designated and (if assessment is desired) assessed for reliability, the PCS coordinates may be used to determine a neurostimulation target for the subject. In certain embodiments, if the PCS coordinates cluster in a similar area, the outliers are dropped and the location of the cluster is designated as the neurostimulation target. In certain embodiments, if the targets cluster in a similar area, drop the outliers, or average out to find a single target region. Different statistical approaches may be used to minimize the effect of outliers, including dropping outliers beyond a certain threshold or transforming the data to a less outlier-sensitive scale (such as a rank transform) before averaging. In certain embodiments, a count of a PCS status is calculated for each portion (by way of example a pixel) of the candidate target region, and one or more sub-regions within the candidate target region having a count above a predetermined threshold are designated as being included within the neurostimulation target. Optionally, the neurostimulation target may consist of one sub-region within the candidate target region.

In certain embodiments, the FCN Targeting method may include instructing a neurostimulation device to apply a neurostimulation procedure to the neurostimulation target. The neurostimulation device may be a transcranial magnetic stimulation (“TMS”) device. The TMS device may include a coil configured to apply accelerated theta-burst stimulation to the neurostimulation target. When TMS is repeatedly applied in a short time frame, it is referred to as repetitive TMS (rTMS). Theta-burst stimulation (TBS) is a patterned form of rTMS, typically administered as triplets of stimulus pulses with 20 ms between each stimulus pulse in the triplet, where the triplet of stimulus pulses is repeated every 200 ms. Intermittent theta-burst stimulation (iTBS) is a form of TBS in which this TBS pattern is interrupted periodically, for example having a repeating pattern of two seconds of TBS and eight seconds of no stimulation, whereas continuous theta-burst stimulation (cTBS) is a continuous delivery of the TBS pattern. Accelerated theta-burst stimulation (aTBS), termed aiTBS for accelerated iTBS and acTBS for accelerated cTBS, is a form of TBS in which multiple sessions are performed per day, for example ten sessions of ten minutes per day having an inter-session interval of 50 minutes, whether delivered on a single day or multiple consecutive or non-consecutive days.

In some variations, the TMS device may be configured to apply TBS, iTBS, and/or cTBS to the neurostimulation target. In other variations, the TMS device may be configured to apply aTBS, aiTBS, and/or acTBS to the neurostimulation target. In other variations, one or more neuromodulation modalities such as without limitation patterns of TMS other than those described herein, transcranial electrical stimulation (tES), transcranial focused ultrasound, epidural stimulation, intracalvarial stimulation, subdural stimulation, intraparenchymal stimulation, intravascular stimulation, and/or focal release of a drug using pumps, drug-eluting materials, drug-coated materials, or molecular cages may be configured to apply neuromodulation to the neurostimulation target instead of or in addition to TMS. In some variations, the neurostimulator may comprise a transducer configured to deliver ultrasound energy to the neurostimulation target. The transducer may be configured to deliver focused ultrasound energy to the neurostimulation target.

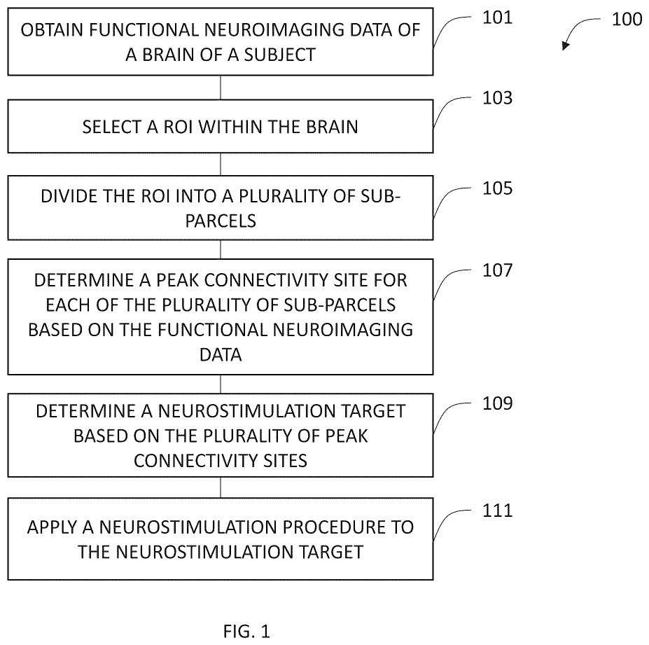

Referring to FIG. 1, a flow chart illustrating an exemplary embodiment of an FCN Targeting method 100 is shown.

In FIG. 1, FCN Targeting method 100 may comprise: a step 101 of obtaining functional neuroimaging data of a brain of the subject, wherein the functional neuroimaging data describes neuronal activation within the brain; a step 103 of selecting a brain region of interest (“ROI”) within the subject's brain; a step 105 of dividing the ROI into a plurality of parcels, and a step 107 of determining a peak connectivity site for each of the plurality of parcels based on the functional neuroimaging data. In certain embodiments, the peak connectivity site for each parcel may be characterized by a pattern of neuronal activity in a portion of a candidate target region having a high degree of synchrony or anti-synchrony of neuronal activity in the corresponding parcel. Certain embodiments of the FCN Targeting method may comprise a step 109 of determining a neurostimulation target based on the plurality of peak connectivity sites. Certain embodiments of the FCN Targeting method may comprise a step 111 of instruction a neurostimulation device to apply a neurostimulation procedure to the neurostimulation target. When FCN Targeting is used to treat depression, the method may include using the dorsal attention network as the ROI, and parcellation as further detailed in Example 1.

### Determining Neurostimulation Targets Based on Weighted Seed-Circuit Pairs

Generating personalized neurostimulation targets may improve treatment outcomes for individuals, as previously mentioned. Currently, there are no objective biomarkers (including imaging findings or lab tests) that can help a clinician choose the right course of neurological or psychiatric treatment for a patient. Multiple initial treatment choices, such as different drugs, can be beneficial for the average patient. As a result, clinicians usually choose initial treatment based on factors other than efficacy. In some cases, these choices may be based on which side effect profile a patient is most likely to tolerate. In other cases, the choices may be based on the clinician's personal preferences such that the clinician often chooses the same initial treatment for most of their patients.

A similar challenge has arisen for clinicians in selecting the transcranial magnetic stimulation (TMS) treatment target. TMS is typically applied to the dorsolateral prefrontal cortex (DLPFC) for treatment of depression. However, the DLPFC is a broad area that contains many different potential targets. Thus, clinicians may also select a target based on personal preference rather than optimizing the target for the patient, oftentimes selecting the same target for every patient. There may be some patients who are more likely to respond to some targets and others who are likely to respond to other targets. There may also be a group of patients who are unlikely to respond to TMS altogether, irrespective of the target.

In clinics that use image-guided TMS targeting, the most common target is a DLPFC site for which resting-state functional magnetic resonance imaging (fMRI) reveals anti-correlation to a targeting seed such as the subgenual cingulate cortex (SGC). This anti-correlation is believed to normalize activity in the SGC. However, not all patients have abnormal activity in this brain region; some patients with major depression are affected in other brain regions, such as the anterior insula or the nucleus accumbens. For these patients, a different treatment target may be more appropriate. Thus, a comprehensive targeting model should consider the connectivity of various brain regions in a patient, and target the region determined to be most suitable.

Accordingly, in some variations, the methods and systems described herein may use an algorithm (e.g., a machine learning algorithm) that analyzes the connectivity of various brain regions by calculating a weight value for a plurality of seed-circuit pairs, and determining a neurostimulation target based on the seed-circuit pair having the greatest weight value. For example, the identification of at least one neurostimulation target may include obtaining functional neuroimaging data of a brain of the subject that describes neuronal activation of a region of interest (ROI) within the brain; selecting at least one seed region within the brain of the subject; and pairing the at least one seed region with a plurality of brain circuits to generate a plurality of seed-circuit pairs. An algorithm may be applied to the plurality of seed-circuit pairs that calculates a weight value for each of the plurality of seed-circuit pairs based on a plurality of criteria (weighting factors). The neurostimulation target may then be determined based on the seed-circuit pair having the greatest weight value.

The number of regions of interest selected may range from one to five. For example, the number of regions of interest may be at least one, at least two, at least three, at least four, or at least five. The region of interest may be selected from the group consisting of the subgenual cingulate cortex, anterior insula, nuclear accumbens, medial prefrontal cortex, and a combination thereof.

The plurality of brain circuits may be associated with a brain network. In some variations, the brain network may be a canonical network or a data-driven network. In some variations, the canonical network may include a dorsal attention network, a ventral attention network, a frontoparietal network, a default mode network, or a cognitive control network.

The plurality of brain networks may comprise at least one, two, three, four, five, or more brain circuits. The plurality of brain networks may include a depression network.

At least one seed region and at least one circuit may be paired together. In some variations, a selected seed region may form pairs with multiple networks to create multiple seed-circuit pairs. In other variations, a selected circuit may form pairs with multiple seed regions to create multiple seed-circuit pairs. In one variation, it may be useful for the seed-circuit pair to include the subgenual cingulate cortex and a depression circuit. The number of seed-circuit pairs may be at least two pairs. In general, the plurality of seed-circuit pairs may range from two seed-circuit pairs to 25 seed-circuit pairs, including all values and sub-ranges therein. For example, the number of seed-circuit pairs may be 2, 3, 4, 5, 6, 7, 8, 9, 10, 11, 12, 13, 14, 15, 16, 17, 18, 19, 20, 21, 22, 23, 24, or 25. In some variations, at least five seed regions and at least five circuits are selected. The at least five seed regions and at least five circuits may be paired such that the number of seed-circuit pairs may be at least 25 seed-circuit pairs.

Functional neuroimaging data may describe neuronal activation within the brain, as previously described herein. The functional neuroimaging data may comprise at least one of functional magnetic resonance imaging (fMRI), functional near infrared spectroscopy (fNIRS), doppler ultrasound, focused ultrasound, diffusion tensor imaging, diffuse optical imaging (e.g. diffuse optical tomography), electroencephalography, and magnetoencephalography. fMRI is an imaging device that may be used to observe functional connectivity within the brain. However, this technology may be cumbersome to use due to its size, weight, and cost. Further, this instrument employs radiation to image the brain which may pose a safety risk for both patients and clinicians. Given this, it would be beneficial to develop a method and system for measuring functional connectivity that is safe, cost-effective, and easy to use. fNIRS is an imaging modality that employs light to measure functional connectivity in the brain. This modality is cost-effective, light, and easy to use. Although fNIRS can capture these signals effectively, this imaging modality cannot measure depth. Without this capacity, a clinician is unable to administer treatment to a defined target. To circumvent this issue, ultrasound, as well as doppler variations, may be used in conjunction with fNIRS to assist a clinician in delivering treatment to the correct location. In another variation, doppler ultrasound may be used to measure functional connectivity. In some instances, this may be beneficial due to the superior temporal resolution of doppler ultrasound relative to other imaging modalities, such as fNIRS.

The functional neuroimaging may be used to determine a connectivity value between the selected seed region and selected network in a given seed-circuit pair. For example, there may be at least 25 connectivity values corresponding to each of the at least 25 seed-circuit pairs. The connectivity value may then be processed using a computer-implemented algorithm that applies a plurality of weighting factors (criteria) to each seed-circuit pair to determine an overall weight value for a given seed-circuit pair. For example, at least two, three, or four weighting factors (criteria) may be applied. The weight values for each of the seed-circuit pairs may then be compared to determine the seed-circuit pair with the overall greatest weight value. The seed-circuit pair with the overall greatest weight value may then be identified and used to determine the target location for neurostimulation.

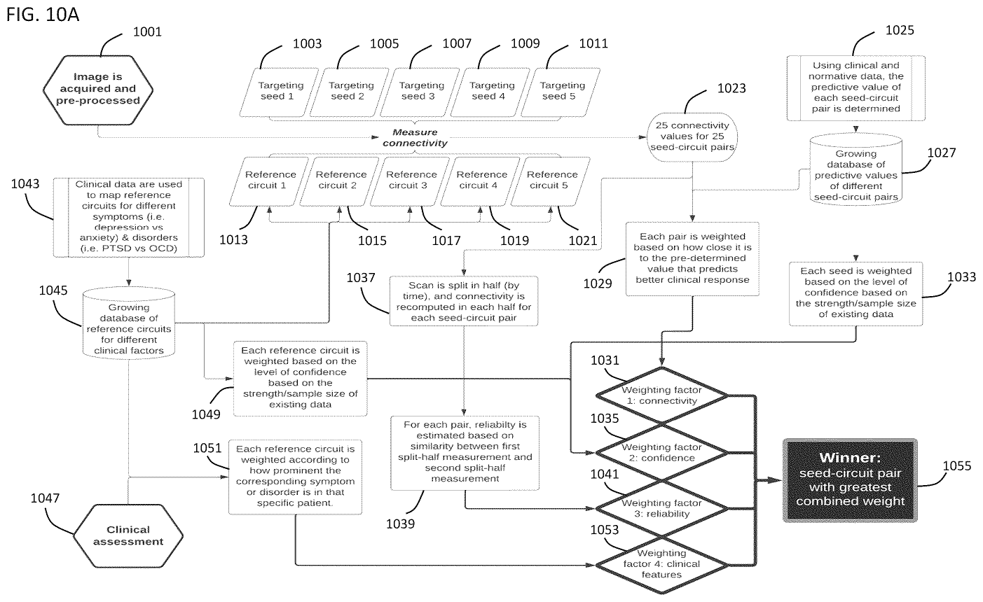

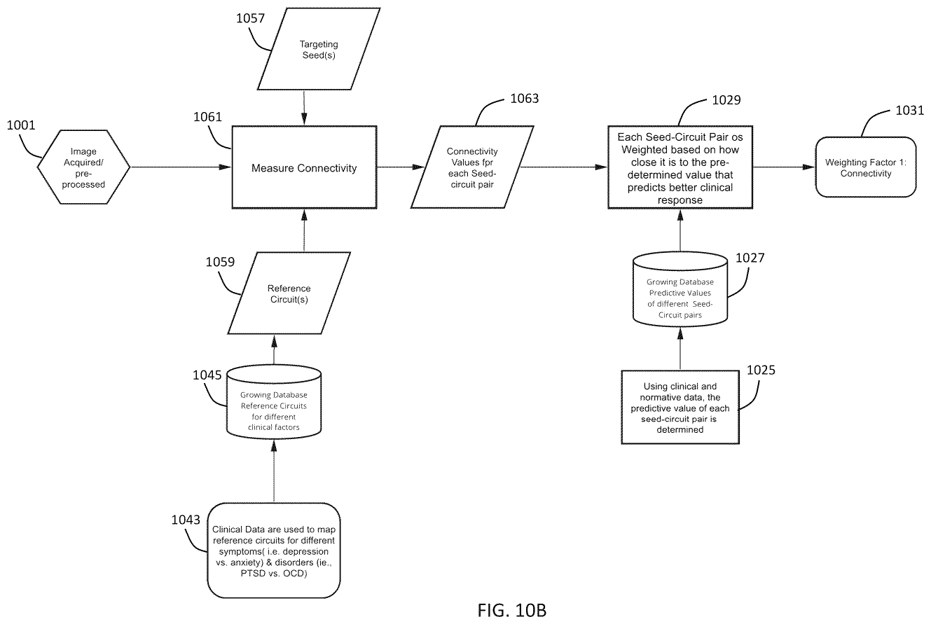

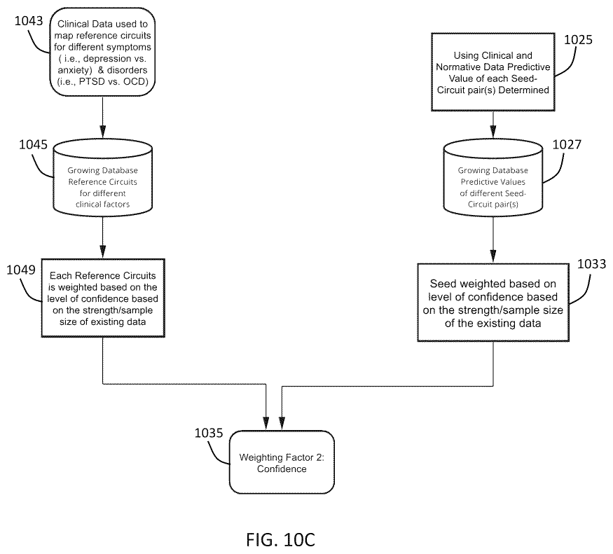

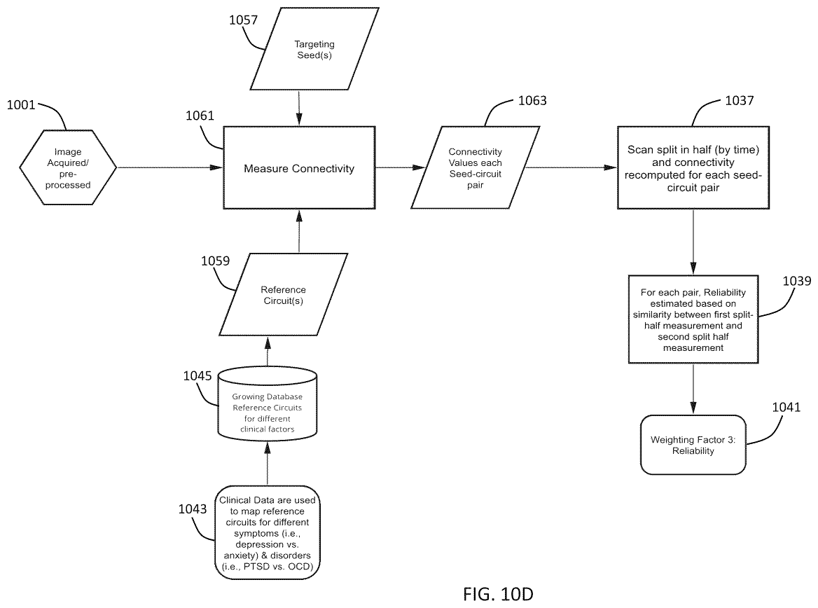

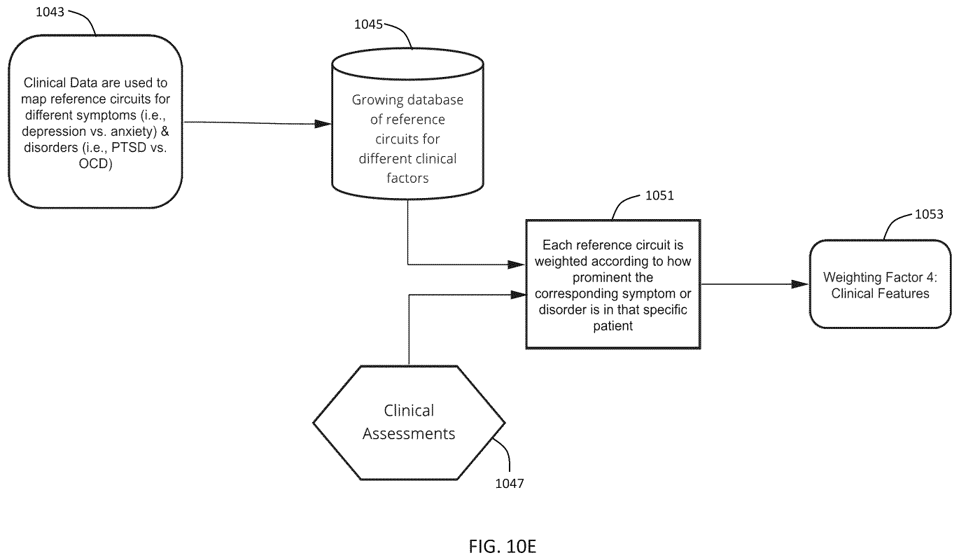

An exemplary embodiment of an algorithm used to identify a neurostimulation target based on weighted seed-circuit pairs is shown in FIGS. 10A-10E. The steps of the method may include acquiring and pre-processing at least one image 1001. In this exemplary embodiment, five targeting seeds 1003, 1005, 1007, 1009, 1011 may be selected. The selected targeting seeds may also be referred to as target seeds. For example, a targeting seed may be the dorsolateral pre-frontal cortex (“DLPFC”). Targeting seeds may be connected to a ROI by reference circuits. In this exemplary embodiment, five reference circuits 1013, 1015, 1017, 1019, 1021 may be selected. The reference circuits may also be associated with brain networks. Each of the five targeting seeds may be paired with each of the five reference circuits to create 25 seed-circuit pairs. The connectivity of each of the seed-circuit pairs may then be measured in step 1023 to determine a connectivity value for each seed-circuit pair.

The connectivity value for each seed-circuit pair may be adjusted using a plurality of weighting factors (criteria) 1031, 1035, 1041, 1053. The plurality of criteria applied to the at least one seed-circuit pair may include a measurement of connectivity between the at least one seed and each of the plurality of brain circuits of the plurality of seed-circuit pairs, a confidence value, one or more clinical features, a reliability value, or combinations thereof.

As shown in FIGS. 10A and 10B, the first weighting factor (criteria) 1031 may be a connectivity value that may be determined for the plurality of seed-circuit pairs. The first weighting factor (criteria) may first depend upon a step 1001 of acquiring and pre-processing at least one image from a neuroimager. Then, in a step 1057 the neuroimaging results may be processed for the at least one target seed. Jointly or separately, in a step 1043 clinical data may be used to map reference circuits for different symptoms and disorders. Then, in a step 1045 the clinical data may be evaluated in view of at least one attribute of at least one database that describes the relationship between a reference circuit and at least one clinical factor. Subsequently, the clinical data and at least one attribute of at least one database may be used to identify at least one reference circuit. In a step 1059, the neuroimaging results may be processed for the at least one reference circuit. Then, at least one target seed may be paired with at least one reference circuit to form at least one seed-circuit pair. In a step 1061, the connectivity of a seed-circuit pair may be measured and, in a step 1063, each seed-circuit pair may be assigned a connectivity value. For example, a larger connectivity value may be assigned to an SGC-depression circuit pair if the neuroimaging results indicate relatively high connectivity between a patient's SGC and the depression circuit. In another example, a larger connectivity value may be assigned to an anterior insula-depression circuit pair if the neuroimaging results indicate relatively high connectivity between a patient's anterior insula and the depression circuit. The connectivity value of the first weighting factor is generally proportional to the magnitude of connectivity between a selected target seed and a reference circuit.

In a step 1029, the connectivity value may be adjusted based on how close the connectivity value is to a pre-determined connectivity value. In a step 1025, the pre-determined connectivity value may be determined for each seed-circuit pair using clinical and normative data. For example, clinical and normative data may be used to calculate a mean and range for the connectivity values observed between a given target and a reference circuit in the general population. In a step 1027, the pre-determined connectivity value may further depend upon at least one attribute of at least one database, wherein the database may contain data that describes a predictable relationship between a target seed and a reference circuit. The pre-determined connectivity value may be established such that the connectivity predicts a positive clinical response to neurostimulation. For example, clinical and normative data may predict a high connectivity value between a hypothetical subgenual cingulate cortex and a hypothetical depression circuit.

As shown in FIGS. 10A and 10C, a second weighting factor 1035 may be a confidence value related to connectivity for each seed-circuit pair. In some variations, the confidence value assigned to a reference circuit may depend upon clinical data that is used to map reference circuits for different symptoms or psychiatric disorders as in a step 1043. The psychiatric disorders may be selected from a group consisting of depression, anxiety, post-traumatic stress disorder, obsessive compulsive disorder, addiction, substance use disorder, bipolar disorder, schizophrenia, and a combination thereof. A psychiatric disorder or symptom may correspond to an identifiable brain circuit or network. For example, symptoms associated with depression may correspond to a depression circuit. In another example, anxiety may correspond to a depression circuit.

As in a step 1045, the second weighting factor 1035 may further depend upon at least one attribute of at least one database that describes a reference circuit as corresponding to certain clinical features. For example, the at least one attribute of a given database may comprise the quantity and quality of the data included in the given database. A greater quantity of supporting data in a given database may proportionally increase the confidence in the database. The quantity of supporting data may be determined by the number of independent studies related to studying the relationship between a reference circuit and a clinical factor. Similarly, the amount of supporting data may be determined by the sample size of a given study. Higher quality data included in a given database may also increase the confidence in the database. The quality of the data may depend upon the conditions in which the data was collected, the entity responsible for collecting the data, the analysis of the data using established or novel principles, and other characteristics of the data. In a step 1049, each reference circuit may then be assigned a weighting value based on the attributes of the clinical data and supporting databases used to map or link a given reference circuit to a given symptom or disorder.

In some variations, the second weighting factor 1035 may separately or jointly depend on clinical and normative data that predict the connectivity between a target seed and a reference circuit in a given seed-circuit pair. As in a step 1025, clinical and normative data may be used to predict the connectivity between a target seed and a reference circuit. For example, clinical data may predict relatively high connectivity in the general population between the subgenual cingulate cortex and the depression circuit. In another example, clinical data may predict relatively high connectivity in the general population between the anterior insula and the depression circuit. Then, in a step 1027, the second weighting factor 1035 may further depend upon at least one attribute of at least one database that describes the predictable relationship between a target seed and a reference circuit. The at least one attribute of a given database may be at least one of the quantity and quality of the data contained within a given database or a plurality of databases. Subsequently, in a step 1033, the confidence value may be based on the predictable relationship between a target seed and a reference circuit and at least one attribute of the plurality of supporting databases.

Furthermore, the second weighting factor 1035 may be based on the results of at least one of steps 1049 and 1033 or a combination thereof. The results of steps 1049 and 1033 may be combined equally or the results of step 1049 may be given more weight than the results of step 1033, or vice versa. Accordingly, the second weighting factor 1035 may quantify the confidence in the connectivity between a target seed and a reference circuit related to a psychiatric or neurological symptom or disorder.

As shown in FIGS. 10A and 10D, a third weighting factor 1041 may be a reliability value assigned to a given seed-circuit pair. Once a seed-circuit pair has been assigned a connectivity value in a step 1063, the neuroimaging scans for the seed-circuit pair may be split in half temporally with respect to the duration of the scan in a step 1037. The scan splitting process may result in a first split-half and a second split-half. The first and second split-halves may independently be assigned a connectivity value. Then, in a step 1039, the connectivity value assigned to the first split-half may be compared to the connectivity value of the second split-half to determine a reliability estimate. The similarity, or lack thereof, between the first and second split-halves may determine the value of the third weighting factor 1041 that is assigned to a given seed-circuit pair or component thereof.

In some variations, the step 1039 may comprise splitting a neuroimaging scan of a seed-circuit pair in half temporally to create a first split-half and a second split-half and comparing the connectivity of the at least one seed-circuit pair in at least one of the first split-half and second split-half to other, previously unselected regions of the brain. For example, the connectivity value from at least one of the first split-half and second split-half may be compared to a connectivity value between a previously unselected region of the brain and at least one of the target seed and reference circuit or combination thereof. In some variations, the previously unselected region of the brain may comprise the entirety of the brain except for the regions associated with the target seed and reference circuit. Accordingly, the third weighting factor 1041 may quantify the reliability of the measured connectivity between a value.

As shown in FIGS. 10A and 10E, a fourth weighting factor 1041 may be determined by a subject's one or more clinical features, if present. The one or more clinical features may comprise a duration of depression, severity of depression, family history of a depressive disorder, history of substance abuse, post-traumatic stress disorder, general anxiety disorder, schizophrenia, obsessive-compulsive disorder, bipolar disorder, or combinations thereof. In a step 1043, the fourth weighting factor 1041 may first depend upon clinical data that is used to map reference circuits to one or more symptoms associated with one or more clinical feature. In a step 1045, the fourth weighting factor 1041 may further depend upon the quantity and quality of databases that describe a given reference circuit and other clinical features. In some instances, the clinical features may include information from clinical assessments such as the MADRS (Montgomery-Asberg Depression Rating Scale) and HAM-D (Hamilton Depression Rating Scale).

The fourth weighting factor 1041 may jointly or separately be based on a step 1047 of at least one clinical assessment of a patient or subject during at least one evaluation by at least one trained medical professional. The clinical assessment may diagnose a patient or subject with any clinical feature and severity thereof. In a step 1051, the at least one clinical assessment may be combined with the clinical data from step 1043 and evaluation of reference databases in step 1045 to determine the prominence in a subject of a given symptom or disorder relative to non-subject specific data. For example, a patient may be diagnosed with depression by a medical professional. The patient's level of depression may be compared to general databases to determine the relative severity or magnitude of the condition against a broader population. Accordingly, the level of depression experienced by the patient may be rated as relatively severe. The level of severity may be further informed by lifestyle conditions of the patient. For example, the subject may be relatively isolated from a social support network, have limited access to hobbies, and may experience other conditions that may contribute to the patient's depression. Therefore, the depression circuit may be assigned a greater value for the fourth weighting factor 1053. In another example, a patient may be diagnosed with an addiction. Accordingly, the corresponding addiction circuit may be assigned a greater value for the fourth weighting factor 1053. In another example, a patient may be diagnosed with severe depression and addiction so both of the depression and addiction circuits may each be assigned a greater value for the fourth weighting factor 1053. Accordingly, the fourth weighting factor 1053 characterizes the clinical features associated with a patient or subject.

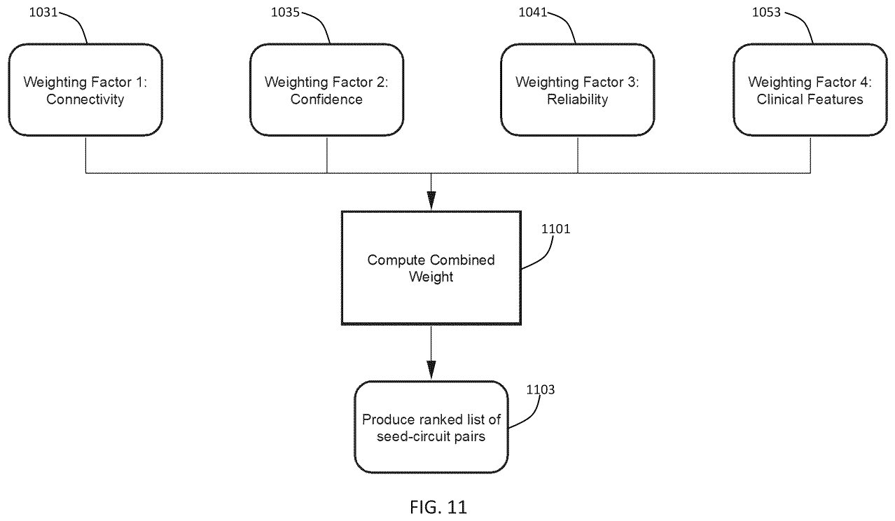

FIG. 11 shows further details of an exemplary method of identifying the seed-circuit pair upon which the neurostimulation target is determined. The first weighting factor (criteria) 1031, second weighting factor (criteria) 1035, third weighting factor (criteria) 1041, and fourth weighting factor (criteria) 1053 may be combined to compute a combined net weight value in a step 1101 for each seed-circuit pair. The combined net weight values assigned to each seed-circuit pair may be ranked according to magnitude. In some instances, the subject may not have any clinical features, and in this case the fourth weighting factor (criteria) 1041 may be omitted from the algorithm. Other weighting factors (criteria) may be omitted from the algorithm as appropriate. In a step 1103, the combined weight values for each seed-circuit pair may be ranked. The neurostimulation target may be determined based on the seed-circuit pair with the largest combined weight value. In some variations, more than one neurostimulation target may be determined.

The target seed in the identified seed-circuit pair may be used to further identify a treatment target based on the resting-state functional connectivity of the target seed to the reference circuit. The reference circuit in the identified seed-circuit pair may be used as a biomarker of treatment response based on functional connectivity to the target seed before, during, and after treatment.

The methods and systems may personalize the target for neurostimulation, which may improve the overall efficacy of the neurostimulation treatment. The method may further include delivering neurostimulation to the determined neurostimulation target. Neurostimulation may be applied by transcranial magnetic stimulation. Transcranial magnetic stimulation (TMS) is a neuropsychiatric therapy where electric pulses are administered to the brain at a region whose activation will cause a desired downstream effect. However, this technology may be cumbersome to use due to the amount of energy that is needed to power high voltage instruments. Further, treating an individual with electrical pulses may pose a safety risk as clinicians and patients are exposed to dangers such as dermal burns and electrocution. Even further, TMS may not have the capacity to stimulate deep brain structures. Thus, in some variations, focused ultrasound (fUS) may be employed to administer neurostimulation treatment. In contrast to using electricity, this technique employs sound to activate desired regions of the brain and has the capacity to stimulate deep brain structures, which may allow clinicians to bypass stimulating cortical target regions. Further, fUS may also be used at multiple target sites, or in conjunction with TMS, to provide multi-targeted neurostimulation therapy.

### Systems

Computer systems that are programmed to implement the FCN Targeting methods are described herein. The systems may include control hardware components, components that receive and process data, and interfaces with a user, etc. For convenience of presentation, a computer system programmed to implement an embodiment of the FCN Targeting method in accordance with the disclosure may be referred to herein as an FCN Targeting system.

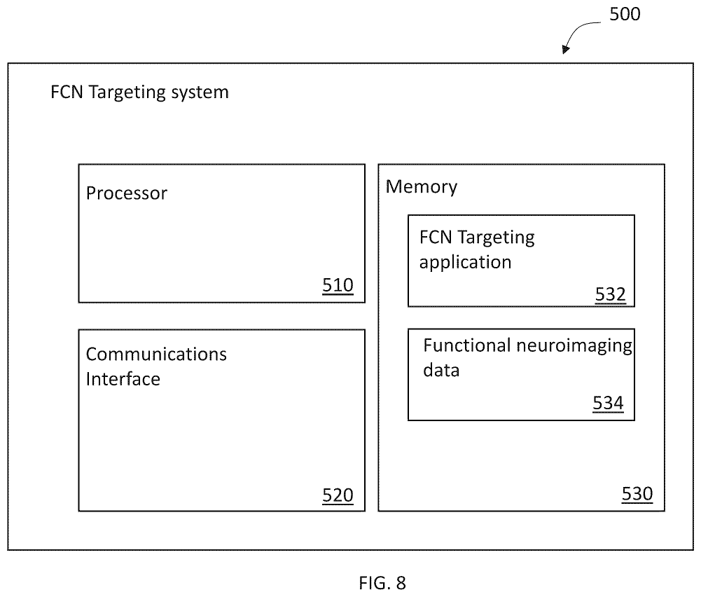

An exemplary FCN Targeting system may be configured as shown in FIG. 8. In FIG. 8, FCN Targeting system 500 may include a processor 510 in communication with a communications interface 520 and a memory 530. In some variations, FCN Targeting systems may comprise multiple processors, multiple memories, and/or multiple communications interfaces. In other variations, components of FCN Targeting systems may be distributed across multiple hardware platforms. Processor 510 may be any type of computational processing unit, including, but not limited to, microprocessors, central processing units, graphical processing units, and parallel processing engines. Communications interface 520 may be utilized to transmit and receive data from other FCN Targeting systems, brain imaging devices, neurostimulation devices, and/or interface devices. Communications interfaces may also include multiple ports and/or communications technologies in order to communicate with various devices as appropriate. Memory 530 may be volatile and/or non-volatile memory. For example, memory 530 may comprise random access memory, read-only memory, hard disk drives, solid-state drives, and flash memory. Memory 530 may store a variety of data, including, but not limited to, functional neuroimaging data 534 and an FCN Targeting application 532 that is stored as a set of computer-readable instructions. In some variations, the FCN Targeting application and/or the neuroimaging data may be received via the communications interface. Processor 510 may be directed by the FCN Targeting application to perform an FCN Targeting method, including, but not limited to, processing functional neuroimaging data and generating neurostimulation targets.

Furthermore, FCN Targeting systems may be implemented on multiple servers within at least one computing system. For example, FCN Targeting systems may be implemented on various remote “cloud” computing systems applications. Other exemplary computing systems include without limitation, personal computers, servers, clusters of computing devices, and/or computing devices incorporated into medical devices.

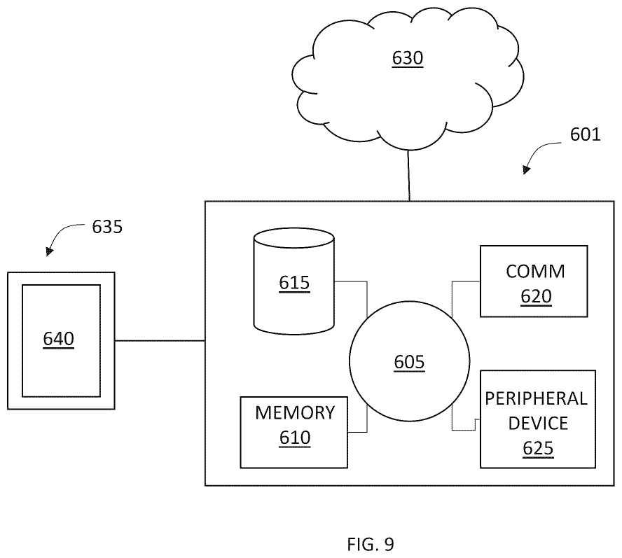

Referring to FIG. 9, an exemplary computer system is shown. In FIG. 9, computer system 601 may be programmed or otherwise configured to execute various aspects of the present disclosure, such as, for example, obtaining functional neuroimaging data of a brain of a subject and determining a neurostimulation target based on the functional neuroimaging data in accordance with embodiments of the disclosure. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The communication interface may be wired or wireless. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 601, may be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a login screen for an administrator to access software programmed to identify a neurostimulation target. Examples of UIs include, without limitation, a graphical user interface (GUI) and web-based user interface.

Methods and systems of the present disclosure may be implemented by way of one or more algorithms. An algorithm may be implemented by way of software upon execution by a central processing unit. For example, the central processing unit may be configured to determine a neurostimulation target by running an algorithm according to the steps illustrated in FIGS. 1 and 10-11 described herein.

Systems for identifying and treating a neurostimulation target are also described herein. The systems generally include a computer programmed to implement one or more embodiments of the above-noted methods. The system may also comprise a communications interface configured to receive data comprising functional neuroimaging data of a brain of the subject, where the functional neuroimaging data describes neuronal activation of a region of interest (ROI) within the brain, a memory storing a set of instructions, and one or more processors that are configured to, responsive to the set of instructions: select a region of interest within the subject's brain; divide the region of interest into a plurality of sub-parcels; determine a peak connectivity site for each of the plurality of sub-parcels based on the functional neuroimaging data, wherein the peak connectivity site is characterized by a pattern of neuronal activity having a high degree of synchrony or anti-synchrony to a pattern of neuronal activity in the corresponding sub-parcel; and determine a neurostimulation target based on the plurality of peak connectivity sites.

In some variations, the one or more processors may be configured to, responsive to the set of instructions, select at least one seed region within the brain of the subject, pair the at least one seed region with a plurality of brain circuits to generate a plurality of seed-circuit pairs, apply an algorithm to the plurality of seed-circuit pairs, wherein the algorithm calculates a weight value for each of the plurality of seed-circuit pairs based on a plurality of criteria, and determine a neurostimulation target based on the seed-circuit pair having the greatest weight value. The system may further comprise a transcranial magnetic stimulation coil configured to deliver neurostimulation to the neurostimulation target. Alternatively, the system may comprise a transducer configured to deliver ultrasound energy to the neurostimulation target. The ultrasound energy may be focused ultrasound energy.

### EXAMPLES

The following examples are illustrative only and should not be construed as limiting the disclosure in any way.

### Example 1: FCN Targeting for Treating Depression

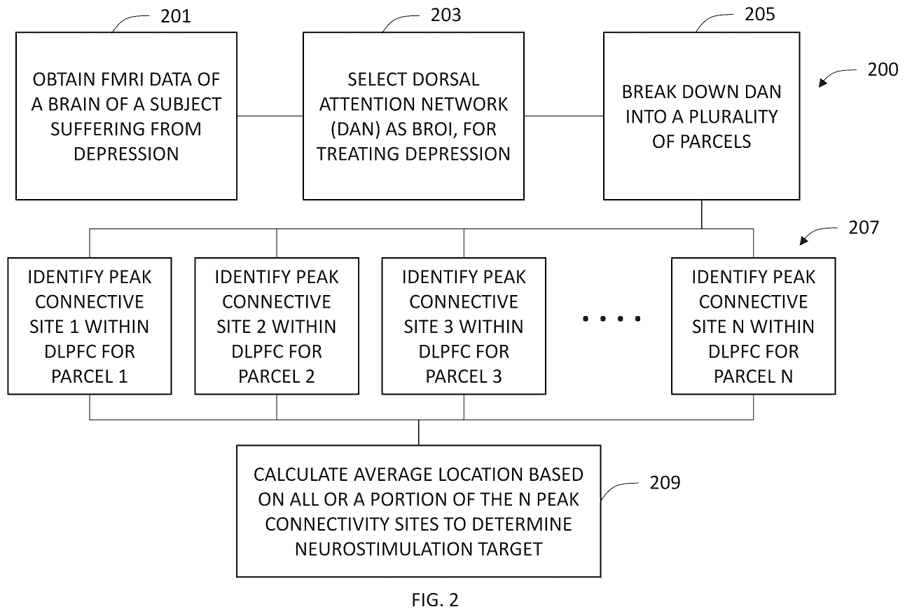

Reference is made to FIG. 2, which shows a flowchart of an exemplary FCN Targeting method 200 for determining a neurostimulation target for treating depression in a subject.

In a step 201, fMRI imaging data obtained from a subject suffering from depression is received.

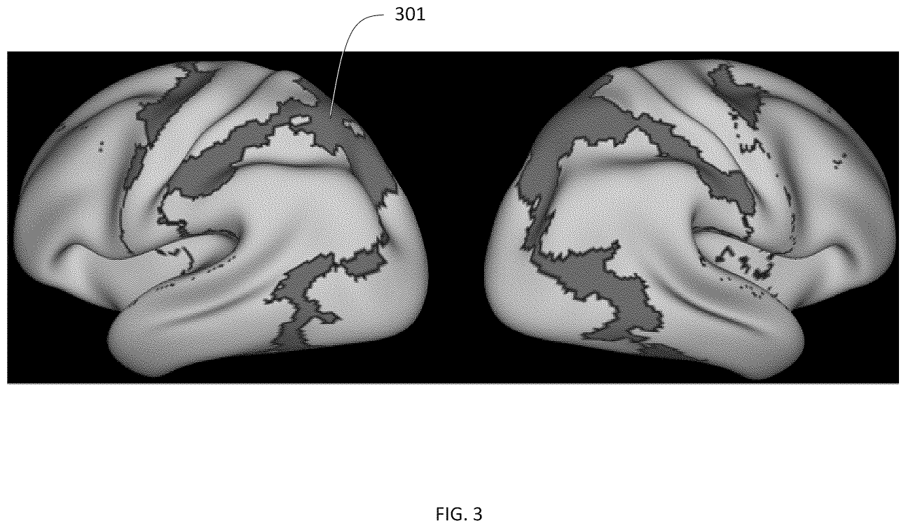

In a step 203, the dorsal attention network (DAN) is designated as a ROI 301 for further analysis based on a previous diagnosis of depression for the subject, and the presumed location of the DAN in the subject is designated. See FIG. 3, which indicates the portion of the subject's brain presumed to be the DAN.

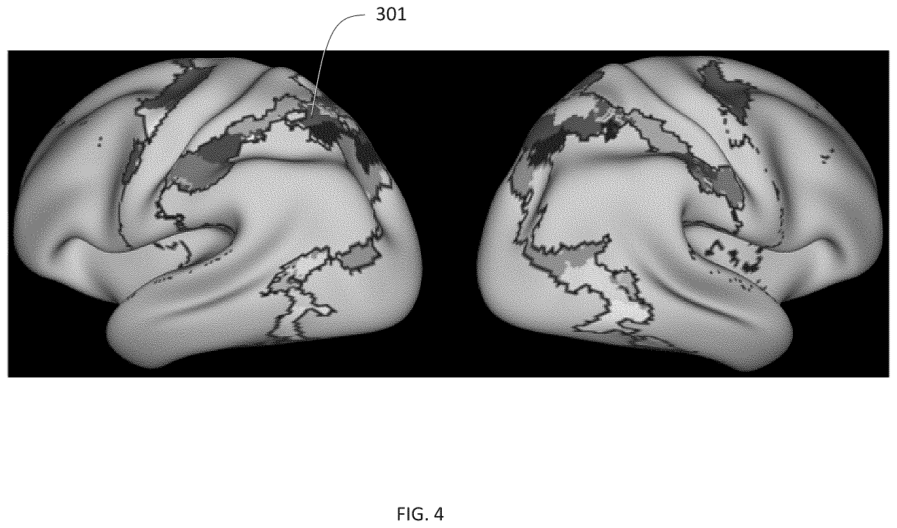

In a step 205, ROI 301 is parcellated into a plurality of parcels based on existing neuroanatomy references, such as the Schaefer 2018 or Gordon 2016 atlases. See FIG. 4, which shows ROI 301 broken up into 50 parcels.

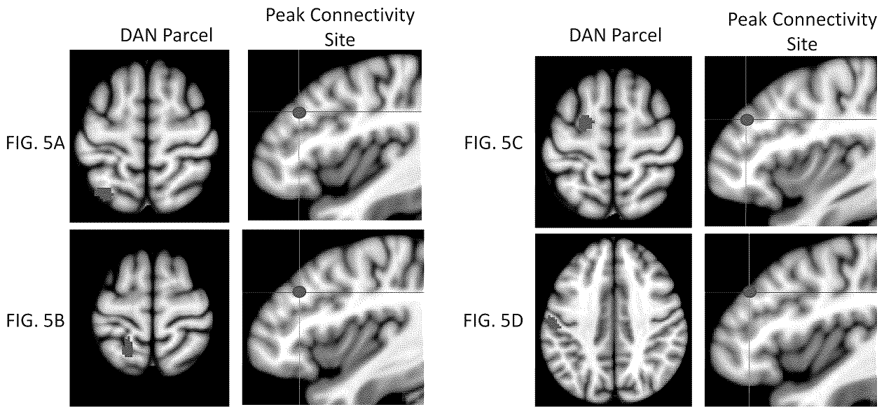

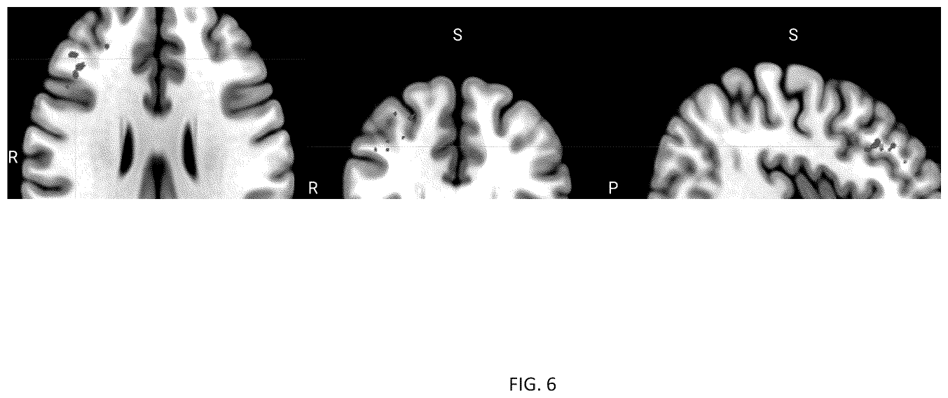

In a step 207, a peak connectivity site is determined within the DLPFC for each of the plurality of parcels parcellated in step 205. The DLPFC is designated as a candidate target region, the synchronicity of neural activity between the DLPFC and each of the parcels of ROI 301, respectively is calculated. For each parcel, the location within the DLPFC where its neural activity is most synchronous with a given parcel of the ROI, in the case of this example a parcel of the DAN, is determined to be the peak connectivity site for the given DAN parcel. Each of FIGS. 5A-5D shows an exemplary DAN parcel (left) and coordinates of the peak connective site (right) corresponding to the given DAN parcel. FIG. 6 shows cross-sectional superior, frontal, and sagittal views of the subject's brain, with a portion of the peak connectivity sites shown in overlay.

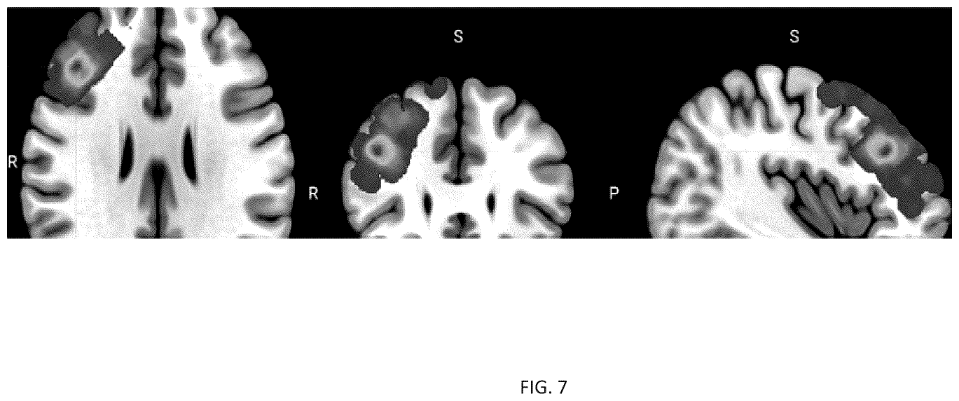

In a step 209, a neurostimulation target based on the plurality of peak connectivity sites as determined in step 207. FIG. 6 shows a cross-sectional superior view (left), frontal view (center) and sagittal view (right) of the subject's brain indicating each connectivity sites determined from each of the respective DAN parcels parcellated in step 205 and shown in FIG. 4. FIG. 7 shows the same cross-sectional views as FIG. 6, but instead of individual peak connectivity sites, shows a heatmap calculated with the plurality of peak connectivity sites. This heatmap was calculated by representing each peak connectivity site as a 3-dimensional smoothing field of intensity values representing a model of the intensity of neuromodulation effect induced by stimulating at exactly that peak connectivity site, in which intensity is maximal at the peak connectivity site itself and decreases with increasing distance from the peak connectivity site itself, reaching an intensity value of zero at a smoothing radius r. Then, each field of intensity values is added together to create the heatmap shown in FIG. 7, in which the central spots of highest intensity (colored red) are designated as the neurostimulation target. The performance of the algorithm can be assessed by observing that there is a single point of highest intensity in each figure, which indicates that the method has converged on one optimal target. In other embodiments, the radius r, shape, or rapidity of fall-off with distance of intensity values in the 3-dimensional smoothing field may be chosen differently or in an asymmetrical shape; for example, to model the known extent of a neuromodulation effect resulting from a specific TMS coil or other transducer used for neurostimulation.

### Example 2: SGC-Depression Circuit Connectivity

The efficacy of SGC-targeted TMS as related to SGC connectivity has previously been demonstrated. The greater the connectivity between the SGC and a previously-published “depression circuit,” the more likely the patient is to improve. There are also several other circuits that may be used instead of the depression circuit, including various resting-state brain networks, as well as a data-driven circuit based on the fact that its connectivity to the SGC may predict clinical outcomes. For example, as shown in studies conducted across 18 participants, the following circuits may also be employed:
A priori depression circuit: SGC connectivity to the a priori depression circuit was correlated with MADRS score at 1 week post-treatment, controlling for baseline MADRS score (r=0.56, p=0.02). This effect survived when additionally controlling for the effect of stimulation site connectivity to the a priori depression circuit (r=0.55, p=0.03). This may be meaningful because the stimulation site is anti-correlated to the SGC, so there is collinearity between these two predictors (r=−0.27). Since the effect survives despite this collinearity, it strongly suggests that the observed effect is truly an effect of SGC connectivity, not stimulation site connectivity.
A priori Yeo networks: It may also be possible to predict clinical outcomes based on SGC connectivity to canonical brain networks (Yeo et al., 2011). Of the seven canonical networks in the consensus Yeo model, four provided significant predictions—dorsal attention network (r=0.56, p=0.02), ventral attention/salience/cingulo-opercular network (r=0.65, p=0.004), frontoparietal control network (r=0.56, p=0.02), and default mode network (r=−0.55, p=0.02). Of note, the limbic network is notably not predictive (r=−0.005, p=0.98), likely because the SGC is already within the limbic network.
Data-driven network: It may also be possible to generate a network target and test it in the same dataset using leave-one-out cross-validation. To generate this network target, the whole-brain connectivity of each participant's individualized subgenual region of interest (ROI) may be mapped and compared to one week post-treatment MADRS, controlling for baseline MADRS. This yields an estimated map of the SGC connectivity profile associated with greater antidepressant efficacy. This estimated map may be generated based on all but one participant, and spatial correlation may be used to compare it to the final participant's SGC connectivity map. Then, spatial correlations may be computed across all participants and may be compared to clinical outcomes. This again shows that SGC connectivity may predict clinical outcomes (r=0.50, p=0.04).
Whole-brain subgenual connectivity: Finally, the overall strength of SGC connectivity may be measured. First, whole-brain SGC connectivity may be mapped. Then, the absolute mean voxel value may be taken of that map. This value may predict clinical outcomes (r=−0.54, p=0.03). Overall strength could also be measured using other metrics, such as the standard deviation of voxel values (r=−0.51, p=0.04), the sum of all anti-correlated values (r=0.61, p=0.009), the peak anti-correlated value (r=0.53, p=0.03), the mean of the strongest 1000 anti-correlated voxels (r=0.62, p=0.007, with similar results when trying different numbers of anti-correlated voxels).
Non-connectivity measures: These predictions may also be made using other imaging measures, including but not limited to regional volume, cortical thickness, cerebral perfusion, fractional anisotropy, mean diffusivity, metabolic activity, or receptor activity.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1.-30. (canceled)

31. A method for identification of a neurostimulation target for a subject, the method comprising:
obtaining functional neuroimaging data of a brain of the subject, wherein the functional neuroimaging data describes neuronal activation of a region of interest (ROI) within the brain;
selecting at least one seed region within the brain of the subject;
pairing the at least one seed region with a plurality of brain circuits to generate a plurality of seed-circuit pairs;
applying an algorithm to the plurality of seed-circuit pairs, wherein the algorithm calculates a weight value for each of the plurality of seed-circuit pairs based on a plurality of criteria; and
determining a neurostimulation target based on the seed-circuit pair having the greatest weight value.

32. The method of claim 31, wherein the functional neuroimaging data comprises data from at least one of functional magnetic resonance imaging (fMRI), functional near infrared spectroscopy (fNTRS), ultrasound, doppler ultrasound, focused ultrasound, diffusion tensor imaging, electroencephalography, and magnetoencephalography.

33. The method of claim 31, wherein the at least one region of interest is selected from the group consisting of the subgenual cingulate cortex, anterior insula, nucleus accumbens, medial prefrontal cortex, and a combination thereof.

34. The method of claim 33, wherein the at least one region of interest comprises the subgenual cingulate cortex.

35. The method of claim 31, wherein at least five regions of interest are selected.

36. The method of claim 31, wherein each of the plurality of brain circuits is associated with a brain network.

37. The method of claim 36, wherein the brain network is a canonical network or a data-driven network.

38. The method of claim 37, wherein the canonical network comprises a dorsal attention network, a ventral attention network, a frontoparietal network, a cognitive control network, or a default mode network.

39. The method of claim 31, wherein the plurality of brain circuits comprises a depression circuit.

40. The method of claim 31, wherein the plurality of brain circuits comprises at least five brain circuits.

41. The method of claim 31, wherein the seed-circuit pair comprises a dorsolateral prefrontal cortex (DLPFC) and a depression circuit.

42. The method of claim 31, wherein the plurality of seed-circuit pairs comprises at least 25 seed-circuit pairs.

43. The method of claim 31, wherein the plurality of criteria comprises a measurement of connectivity between the at least one seed region and each of the plurality of brain circuits of the plurality of seed-circuit pairs, a confidence value, one or more clinical features, a reliability value, or combinations thereof.

44. The method of claim 31, wherein the one or more clinical features comprises a duration of depression, severity of depression, family history of a depressive disorder, history of substance abuse, post-traumatic stress disorder, general anxiety disorder, schizophrenia, obsessive-compulsive disorder, bipolar disorder, or combinations thereof.

45. The method of claim 31, further comprising delivering neurostimulation to the neurostimulation target.

46. The method of claim 45, wherein the neurostimulation is used to treat a psychiatric disorder selected from the group consisting of depression, anxiety, post-traumatic stress disorder, obsessive compulsive disorder, addiction, substance use disorder, bipolar disorder, schizophrenia, and a combination thereof.

47. The method of claim 45, wherein the neurostimulation is used to treat a neurological disorder selected from the group consisting of Parkinson's disease, essential tremor, stroke, epilepsy, traumatic brain injury, migraine headache, cluster headache, chronic pain, consequences of stroke, and combinations thereof.

48. A system for identifying a neurostimulation target for a subject, the system comprising:
a communications interface configured to receive data comprising functional neuroimaging data of a brain of the subject, wherein the functional neuroimaging data describes neuronal activation of a region of interest (ROI) within the brain;
a memory storing a set of instructions;
one or more processors that are configured to, responsive to the set of instructions:
select at least one seed region within the brain of the subject;
pair the at least one seed region with a plurality of brain circuits to generate a plurality of seed-circuit pairs;
apply an algorithm to the plurality of seed-circuit pairs, wherein the algorithm calculates a weight value for each of the plurality of seed-circuit pairs based on a plurality of criteria; and
determine a neurostimulation target based on the seed-circuit pair having the greatest weight value.

49. The system of claim 48, wherein the functional neuroimaging data comprises data from at least one of functional magnetic resonance imaging (fMRI), functional near infrared spectroscopy (fNTRS), ultrasound, doppler ultrasound, focused ultrasound, diffusion tensor imaging, electroencephalography, and magnetoencephalography.

50. The system of claim 48, wherein the at least one region of interest is selected from the group consisting of the subgenual cingulate cortex, anterior insula, nucleus accumbens, and a combination thereof.

51-64. (canceled)
